(19) United States
(12) Patent Application Publication
(10) Pub. No.: US 2014/0378754 A1
(43) Pub. Date: Dec. 25, 2014

(54) UTERINE EMBRYO RETRIEVAL

(71) Applicant: PREVIVO GENETICS, LLC, Piedmont, CA (US)

(72) Inventors: John E. Buster, Providence, RI (US); Moses Cesario, Piedmont, CA (US); Steven Paul Woodard, Cupertino, CA (US)

(21) Appl. No.: 13/924,517

(22) Filed: Jun. 21, 2013

(51) Int. Cl. A61B 17/435

(52) U.S. Cl. CPC A61B 17/435; A61B 2017/4216
USPC 600/34

(57) ABSTRACT

A device for recovering one or more blastocysts from a uterus of a human includes an outer guide member for insertion into a cervical canal of the human. The outer guide member includes a distal portion with an activatable seal for isolating the uterus from the external environment. The outer guide member defines a lumen having a longitudinal axis. The device also includes an inner catheter located within the lumen and slidable along the longitudinal axis of the lumen relative to the outer guide member. The inner catheter has a distal tip positionable distally of the seal to extend into the uterus. The distal tip includes a semi-permeable absorbent head. The device defines a distal suction port for aspirating blastocysts from the uterus through the absorbent head.

This disclosure is related to U.S. patent application Ser. No. 13/335,170, filed Dec. 22, 2011, titled “RECOVERY AND PROCESSING OF HUMAN EMBRYOS FORMED IN VIVO,” hereby incorporated by reference in its entirety.

### TECHNICAL FIELD

This disclosure relates to uterine embryo retrieval.

### BACKGROUND

Recovery and re-implantation of human embryos from human subjects have been performed for the past three decades. In particular, in-vivo fertilized embryos have been recovered from fertile women and transferred to infertile recipient women, producing donor-to-recipient transplanted human pregnancies. The first reported procedure was performed by a University of Los Angeles team in 1983 and produced a live birth in 1984.

### SUMMARY

In general, in an aspect, at a time when a woman's uterus contains in vivo fertilized preimplantation blastocysts, a seal is provided, between the uterus and the external environment, against flow of fluid from the uterus to the external environment. While the seal is provided, fluid is delivered past the seal and into the uterus. The delivered fluid is withdrawn, with the blastocysts, past the seal and from the uterus to the external environment.

Implementations may include one or more of the following features. The recovered in vivo pre-implantation blastocysts are recovered for genetic diagnosis or genetic therapy or sex determination or any combination of two or more of them. One or more of the blastocysts are returned to the uterus of the woman. The one or more blastocysts are returned to the uterus of the woman without having frozen the blastocysts. The blastocysts resulted from artificial insemination. The blastocysts resulted from causing superovulation in the woman. At least one of the pre-implantation blastocysts is treated. The treating includes gene therapy. The in vivo fertilized preimplantation blastocysts are withdrawn from the uterus with an efficiency of greater than 50%. The in vivo fertilized preimplantation blastocysts are withdrawn from the uterus with an efficiency of greater than 80%. The in vivo fertilized preimplantation blastocysts are withdrawn from the uterus with an efficiency of greater than 90%. The in vivo fertilized preimplantation blastocysts are withdrawn from the uterus with an efficiency of greater than 95%. The embryos are frozen. The delivering or withdrawing or both of the fluid is pulsatile. The fluid is withdrawn while the seal is being provided. The seal enables essentially all of the fluid to be withdrawn. The withdrawing of fluid includes aspirating the fluid from the uterus. Both the delivering and the withdrawing are pulsatile and the pulses of the delivering of the fluid and of the withdrawing of the fluid are coordinated.

In one general aspect, a device for recovering one or more blastocysts from a uterus of a human includes an outer guide member for insertion into a cervical canal of the human. The outer guide member includes a distal portion with an activatable seal for isolating the uterus from the external environment. The outer guide member defines a lumen having a longitudinal axis. The device also includes an inner catheter located within the lumen and slidable along the longitudinal axis of the lumen relative to the outer guide member. The inner catheter has a distal tip positionable distally of the seal to extend into the uterus. The distal tip includes a semi-permeable absorbent head. The device defines a distal suction port for aspirating blastocysts from the uterus through the absorbent head.

Implementations may include one or more of the following features. For example, the inner catheter may define the distal suction port. The absorbent head may have an atraumatic shape. The inner catheter may be steerable. The inner catheter may include a memory-retaining material. Upon extension into the uterus, the inner catheter may be configured to be steered by bending according to a pre-bent shape of the memory-retaining material. The activatable seal may be a balloon collar. The activatable seal may be an expandable foam.

In another general aspect, a system for recovering one or more blastocysts from a uterus of a human includes a device and a controller. The device includes an outer guide member for insertion into a cervical canal of the human. The outer guide member includes a distal portion with an activatable seal for isolating the uterus from the external environment. The outer guide member defines a lumen having a longitudinal axis. The device also includes an inner catheter located within the lumen and slidable along the longitudinal axis of the lumen relative to the outer guide member. The inner catheter has a distal tip positionable distally of the seal to extend into the uterus. The distal tip includes a semi-permeable absorbent head. The device defines a distal suction port for aspirating blastocysts from the uterus through the absorbent head. The controller is programmed to apply vacuum to the device from a vacuum source remote from the device.

Implementations may include one or more of the following features. For example, the controller may include a pump for applying the vacuum. The controller may include electro-mechanical means for controlling the vacuum. The system may include an embryo recovery trap for receiving the blastocysts.

In another general aspect, process for recovering one or more blastocysts from a uterus of a human includes placing a device trans-vaginally into the cervical canal. The device includes an outer guide member and an inner catheter located within the outer guide member. The outer guide member includes a seal for isolating the uterus from the external environment. The process also includes advancing the inner catheter relative to the outer guide member positioning a distal region of the inner catheter within the uterus and applying a vacuum to the uterus to aspirate blastocysts from the uterus through an absorbent head of the inner catheter.

Implementations may include one or more of the following features. For example, placing the device may include locating the seal in the cervical canal. Locating the seal ,ay include locating the seal between the internal cervical os and the external cervical os such that the seal does not extend into the vagina or the uterus. Advancing the inner catheter may include swabbing an inner surface of the uterus with the absorbent head. Advancing the inner catheter may include positioning the absorbent head proximate an inner wall of the uterus.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a side view of a uterine retrieval device within a female reproductive tract.

FIG. 2 is a side view of the uterine retrieval device.

FIGS. 3a and 3b are cross-sectional views of portions of the uterine retrieval device.

FIG. 4 is a close-up perspective view of a distal portion of the uterine retrieval device.

FIG. 5 is a side cross-sectional view of a handle portion of the uterine retrieval device.

FIG. 6 is a side view of the uterine retrieval device in a refracted position.

FIG. 7 is a side view of the uterine retrieval device in an extended position.

FIG. 8 illustrates a steerable tip of the uterine retrieval device in FIG. 7.

FIG. 9 is a perspective view of a distal portion of the uterine retrieval device.

FIG. 10 is a partially cut side view of the uterine retrieval device.

FIG. 11 is a side view of the uterine retrieval device.

FIG. 12 is a front view of a cervical stop of the uterine retrieval device.

FIGS. 13-15 are partially cut side views of the uterine retrieval device.

FIG. 16 illustrates the uterine retrieval device connected to a control cart.

FIG. 17 is a flow chart illustrating an example process that uses a uterine retrieval system.

FIGS. 18-30 illustrate a uterine retrieval process using the uterine retrieval device.

FIGS. 31 and 32 are side views of another alternative implementation of the uterine retrieval device.

FIGS. 33 and 34 illustrate another alternative uterine retrieval process using the uterine retrieval device of FIGS. 31 and 32.

FIG. 35 is a side view of an alternative implementation of the uterine retrieval device.

FIG. 36 is a side view of another alternative implementation of the uterine retrieval device.

Like reference symbols in the various drawings indicate like elements.

### DETAILED DESCRIPTION

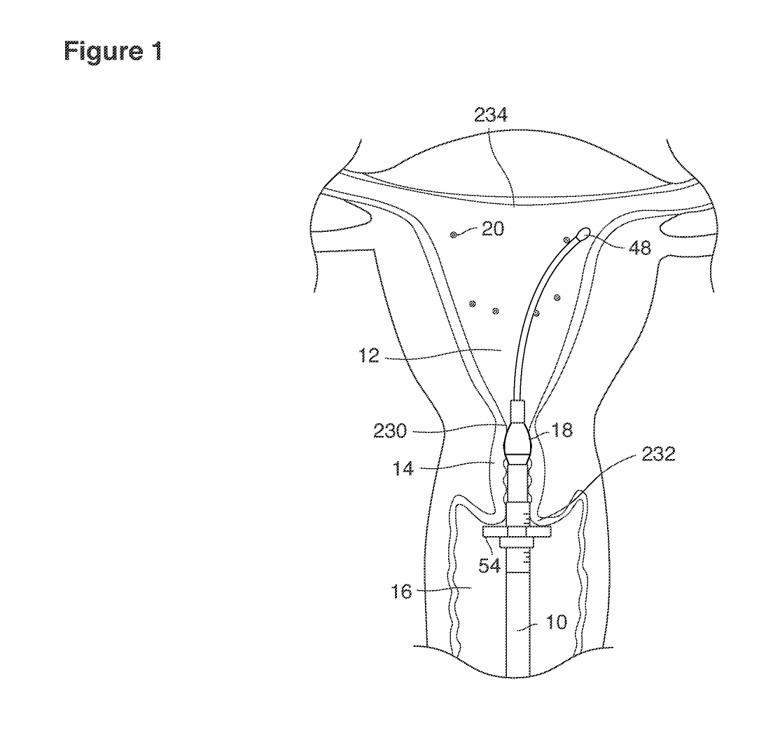

Uterine embryo retrieval is performed to withdraw in vivo fertilized preimplantation embryos from a woman. The preimplantation embryos are produced, for example, by superovulation and artificial insemination. Referring to FIG. 1, to perform the uterine retrieval, a uterine retrieval device 10 is inserted into the uterine cavity 12 via the cervical canal 14 and the vagina 16. The uterine cavity 12 can be sealed from the external environment by an activatable seal, for example, an inflatable balloon collar 18 of the uterine retrieval device 10, and uterine retrieval is performed by withdrawing preimplantation embryos, i.e., blastocysts 20, from the uterine cavity 12.

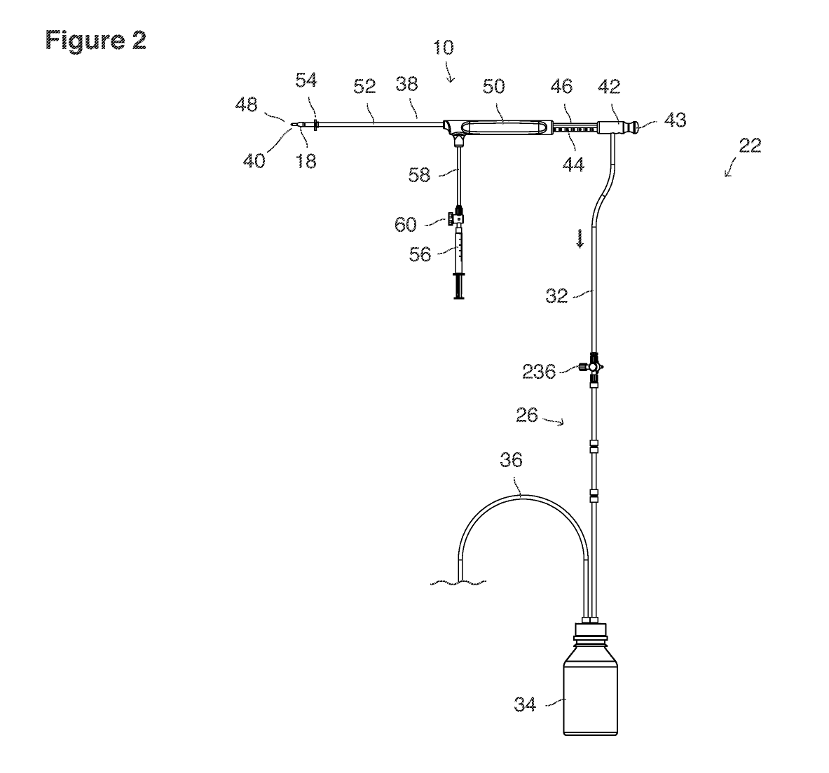

Referring to FIG. 2, a uterine retrieval system 22 includes the uterine retrieval device 10, including an outflow section 26. The outflow section 26 includes a suction recovery line 32 attached to an embryo recovery trap 34, which is attached to a suction line 36. The uterine retrieval device 10 includes an outer, formable guide member 38 and an inner catheter 40 slidably received within the outer guide member 38.

The inner catheter 40 includes a manifold 42 to which the suction recovery line 32 is attached. The manifold 42 has a control knob 43 for manipulating the inner catheter 40, and extending distally from the manifold 42, the inner catheter 40 includes a stabilizing bar 44, a suction line 46, and an absorbent tip 48. The tip 48 can have an atraumatic shape to, for example, help reduce injury during insertion of the tip 48 into the uterine cavity 12. The tip 48 can be made from one or more semi-permeable materials, such as polyamide, among others, to help absorb blastocysts 20 through a sponge-like effect, as discussed further below. The outer guide member 38 includes a handle 50, a guide arm 52, a cervical stop 54, and a seal, for example, the balloon collar 18. The balloon collar 18 is inflated using air or liquid delivered by a supply syringe 56 through a supply line 58 attached to the handle 50. Fluid flow through supply line 58 is controlled by a stopcock 60.

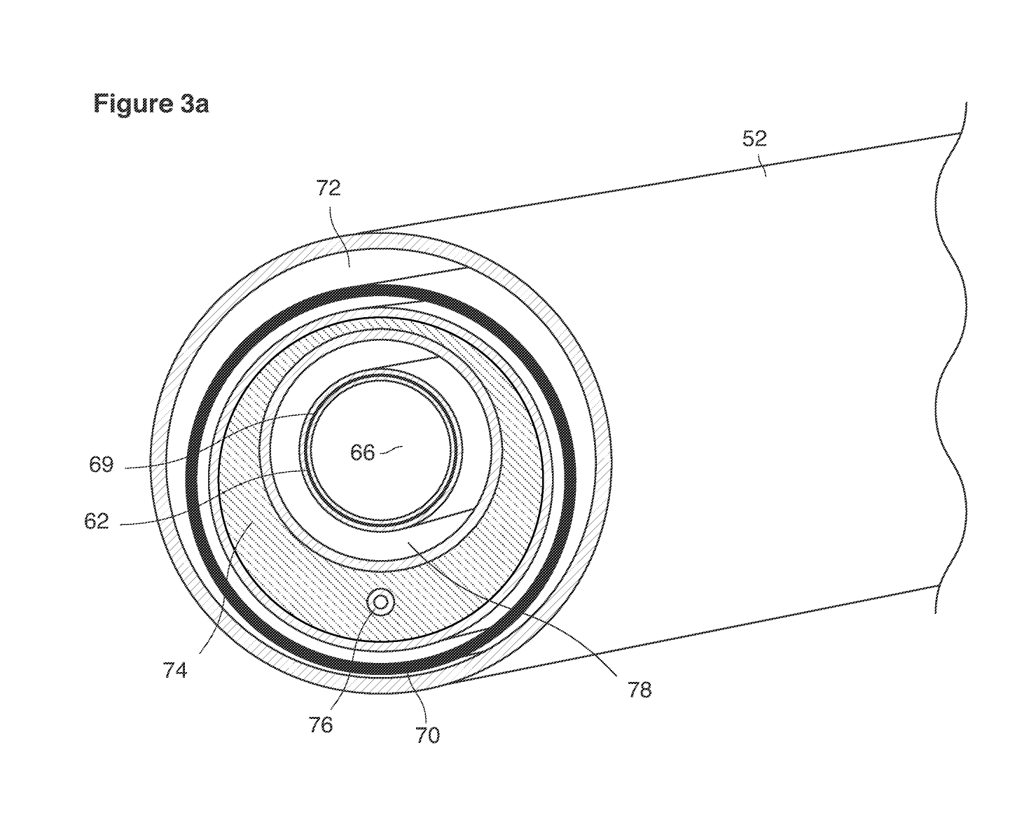

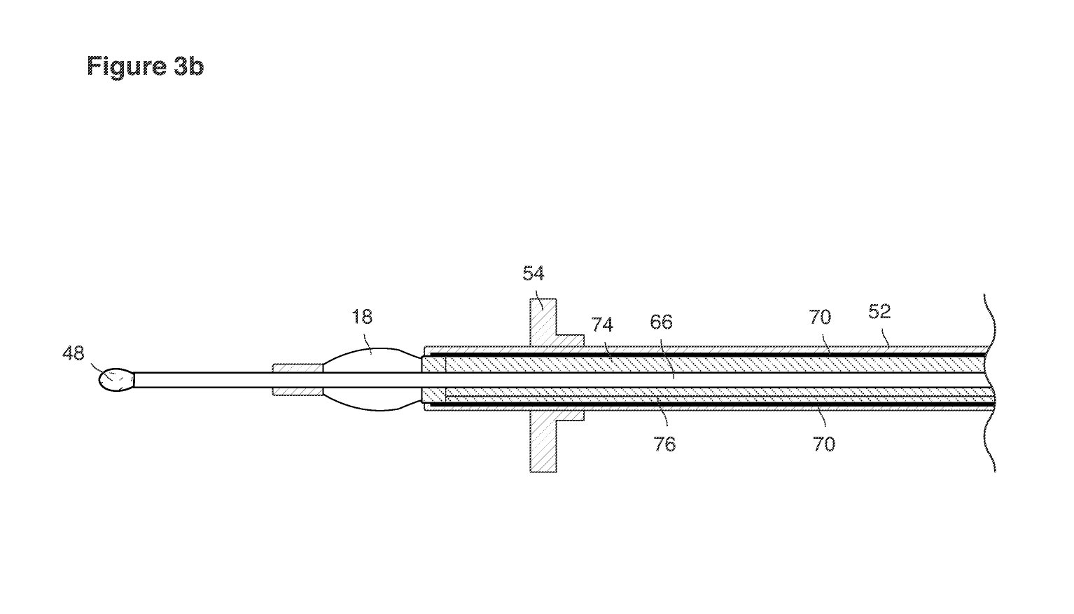

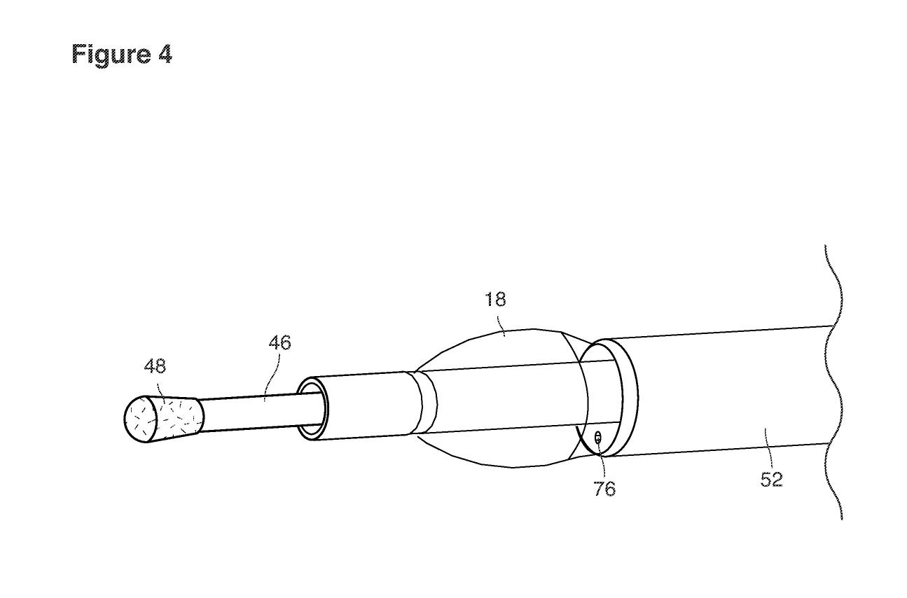

Referring to FIGS. 3A and 3B, the suction line 46 of the inner catheter 40 can be a tubular member 62. The tubular member 62 defines an outflow lumen 66 for removal blastocysts from the uterine cavity. The tubular member 62 includes a formable member 69 that allows the tubular member 62 to be manipulated within the uterine cavity 12, as discussed further below. The outer guide member 38 includes a formable tube 70 located within a lumen 72 of the guide arm 52. The formable tube 70 surrounds a support member 74, which defines a lumen 76 connected to the balloon inflation supply line 58. FIG. 4 shows the termination of the supply lumen 76 at the balloon collar 18. Support member 74 defines a lumen 78 (FIG. 3A) that receives the suction line 46 of the inner catheter 40.

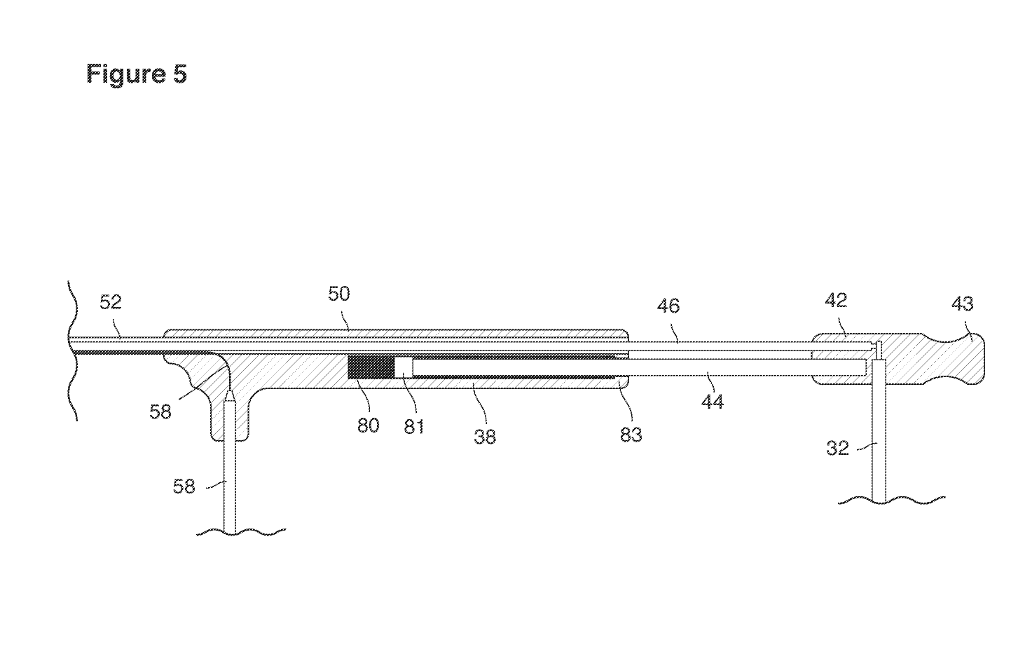

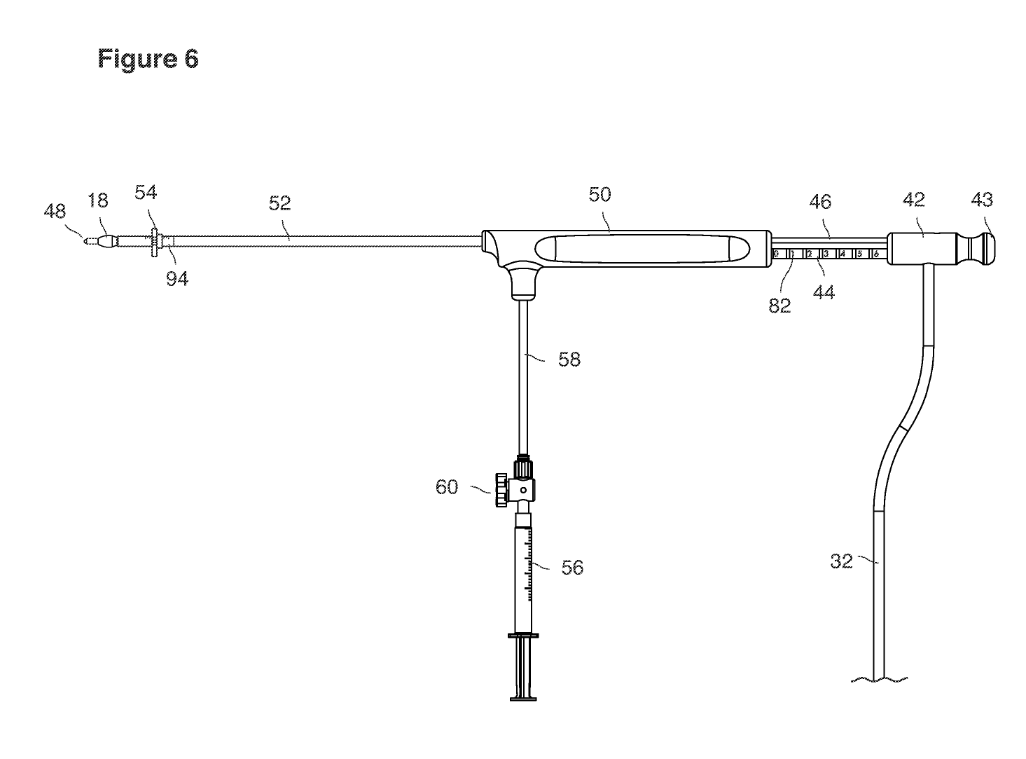

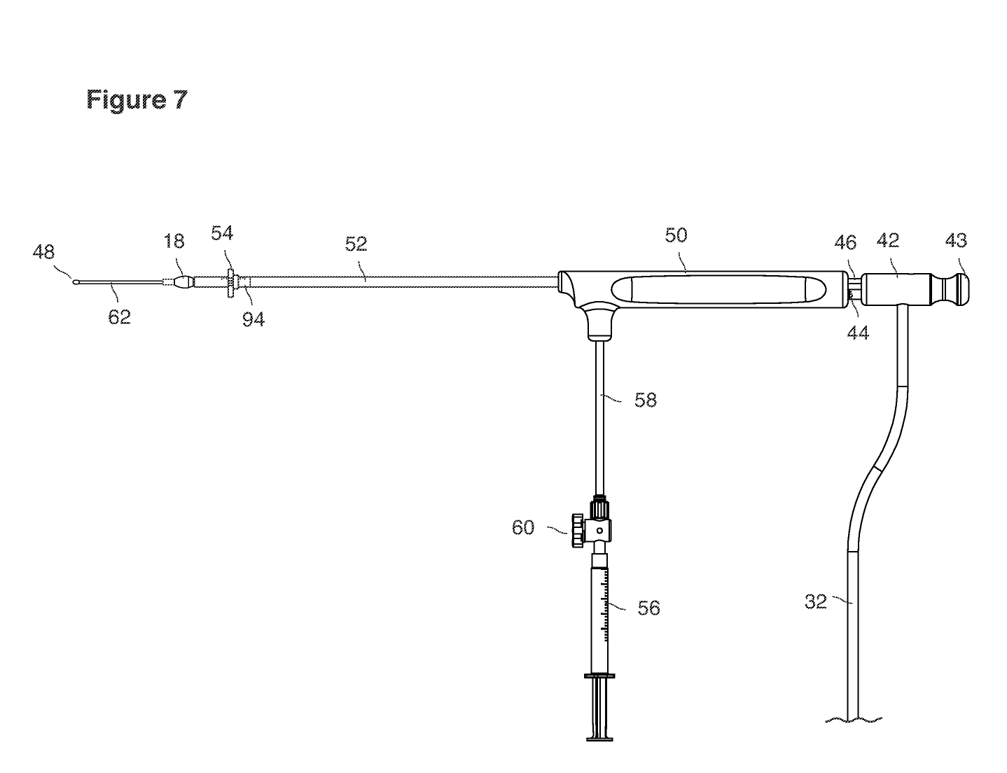

Referring to FIG. 5, the handle 50 defines a slot 80 that receives the stabilizing bar 44. When the inner catheter 40 slides axially relative to the outer guide member 38, the stabilizing bar 44 slides along the slot 80. The stabilizing bar 44 helps support the manifold 42. As illustrated in FIG. 6, the stabilizing bar 44 includes indicia 82 that indicate the extent of insertion of the inner catheter 40 relative to the outer guide member 38. The inner catheter 40 can be moved axially between the retracted position of FIG. 6, and the extended position of FIG. 7. The stabilizing bar 44 terminates in a head 81 and the handle 50 includes a stop 83 which prevents the head 81 from exiting from the slot 80 such that the inner catheter 40 and the outer guide member 38 are permanently joined to form a single, integrated device, i.e., the suction line 46 cannot be completely removed from the outer guide member 38 by the operator.

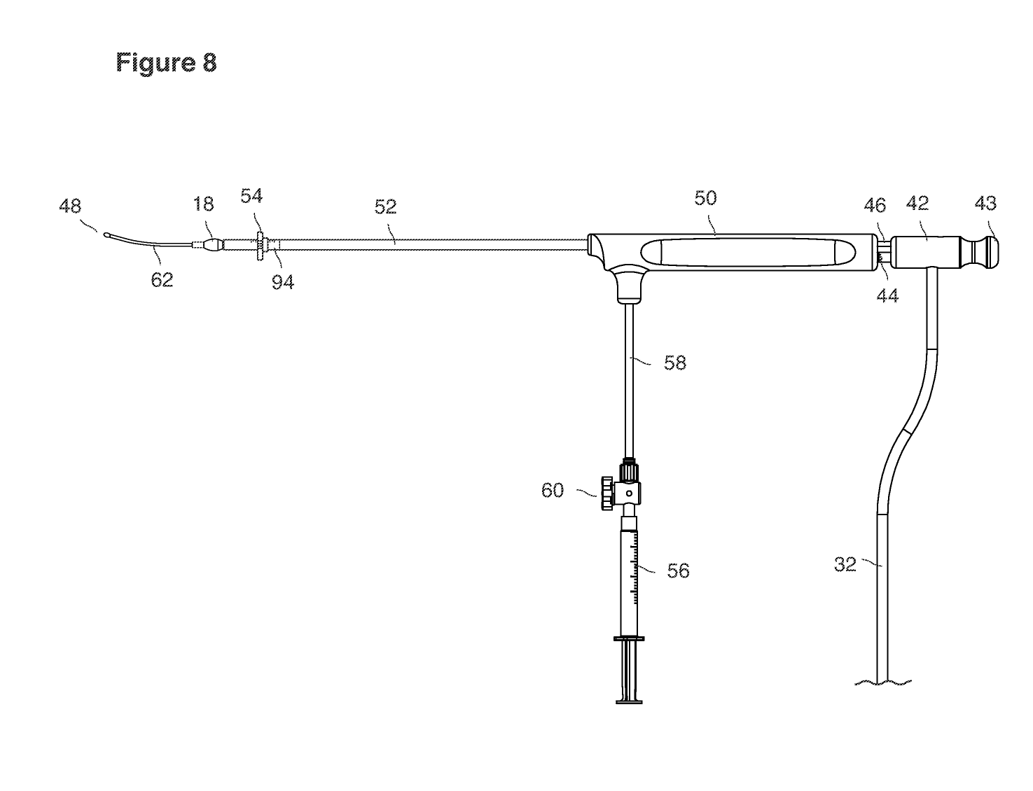

Referring to FIG. 8, the outer tubular member 62, due to the formable member 69 (FIG. 3a), can bend into a pre-shaped curve as the inner catheter 40 is extended. For example, the outer tubular member 62 can be pre-shaped according to the particular shape of a woman's uterus. Accordingly, a path that the absorbent tip 48 takes as it extends into the uterine cavity can be preset, for example, so that it follows the contoured surface of the uterine wall. Additional intrauterine manipulation of the absorbent tip 48 may be accomplished by rotating the inner catheter 40 while extending the inner catheter 40. In some cases, the handle 50 may include a wheel to help rotate the inner catheter 40 relative to the handle 50. Alternatively, or additionally, the entire device 10 may be rotated. In some cases, the formable member 69 may include steering wires that allow the user to actively steer the absorbent tip 48.

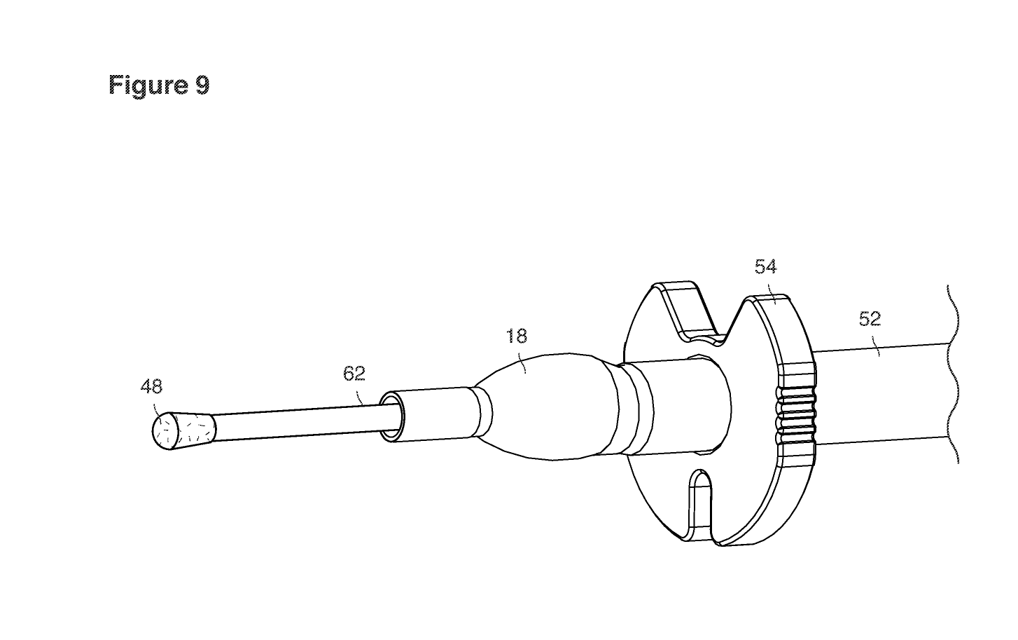

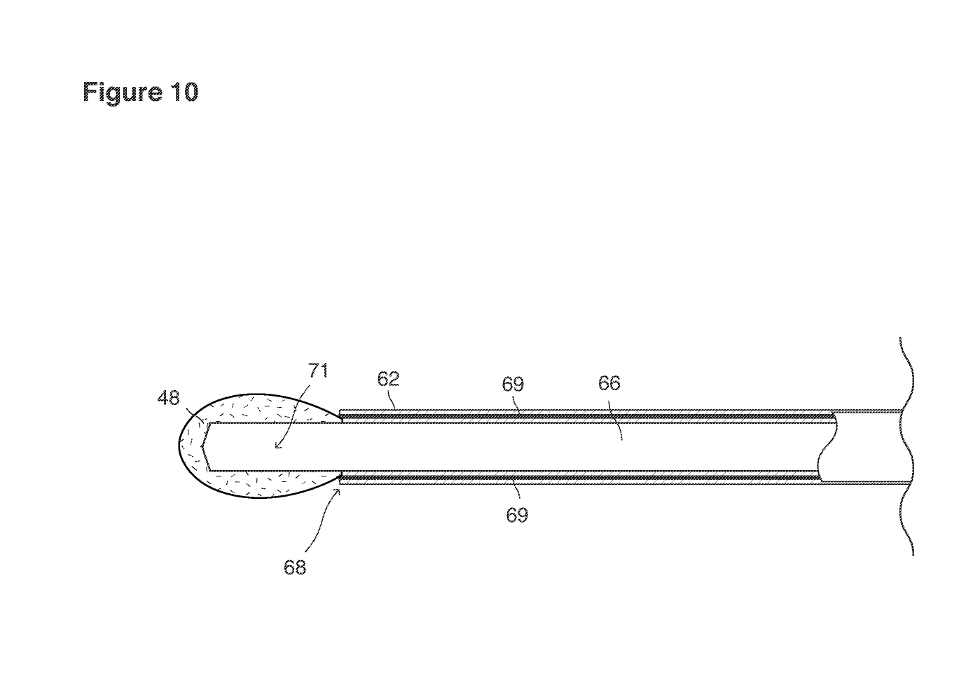

Referring to FIGS. 9-11, the tubular member 62 of the suction line 46 terminates at a distal end 68 and is attached to the absorbent tip 48, for example, via one or more of heat weld, adhesive, and extrusion bonding. In some cases, a portion of the distal end 68 may extend into the tip 48 to help secure the member 62 to the tip 48. The outflow lumen 66, which is in fluidic contact with the absorbent tip 48, can aspirate blastocysts 20, including any fluid that may be present and entraining the blastocysts 20, away from the tip 48 toward the embryo recovery trap 34 (FIG. 2). As shown in FIG. 10, an inside region of the absorbent tip 48 can define a volume 71 that fluidically connects with the outflow lumen 66 to help carry blastocysts 20 away from the tip 48. In some cases, the volume 71 of the absorbent tip 48 may receive a portion of the tubular member 62 or a tubular extension thereof.

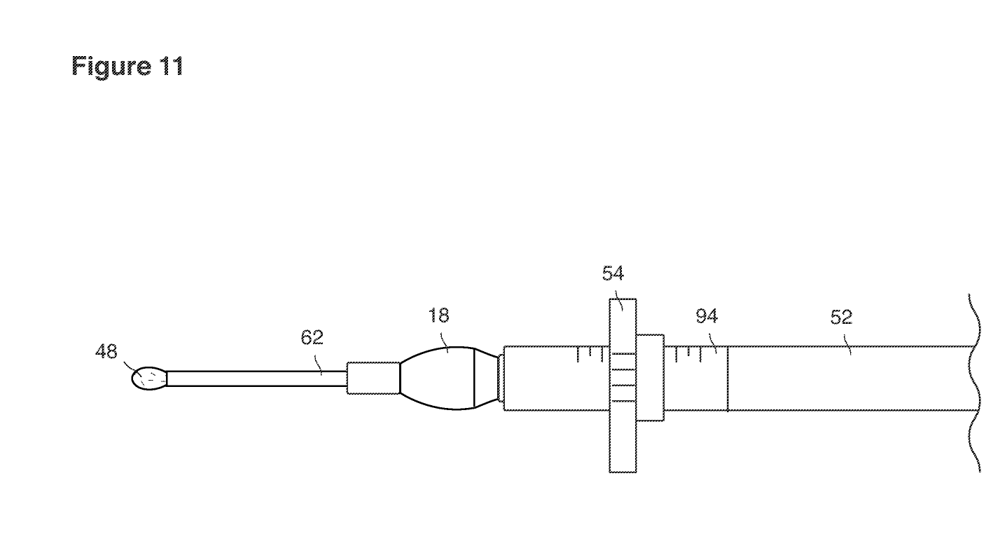

The position of the cervical stop 54 is adjustable relative to the balloon collar 18 along a cervical stop scale 94 (FIG. 11) on the guide arm 52. The position of the cervical stop 54 defines a dimension corresponding to a distance from an opening of the cervix at the vagina (the external cervical os) and an opening of the cervix at the uterus (the internal cervical os). The cervical stop 54 can be clamped in a set position along the guide arm 52.

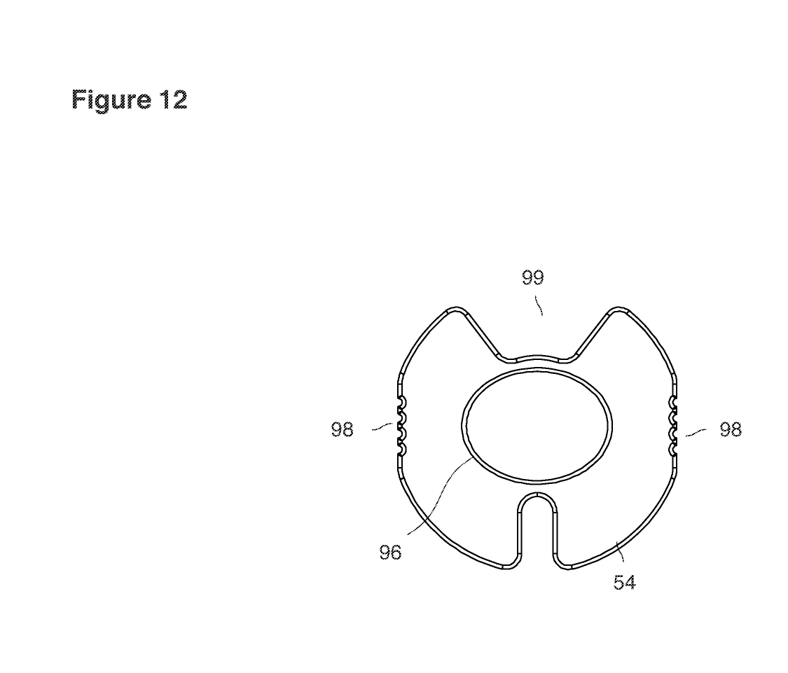

Referring to FIG. 12, the cervical stop 54 includes a locking ring 96 and flange adjustment grips 98. In its rest state, the locking ring 96 is not circular in shape and has an inner dimension smaller than the outer diameter of the guide arm 52 to lock the cervical stop 54 in position. By squeezing in on the flange adjustment grips 98, the operator can deform the shape of the locking ring 96 to a more circular shape that can slide along the guide arm 52 to adjust the position of the cervical stop 54. Upon release of the squeezing force, the locking ring 96 returns toward it rest state, locking the cervical stop 54 in place. The cervical stop 54 is shaped to have a visual port 99 that allows the operator to see the cervix and align the absorbent tip 48 during insertion of the uterine device 10. The cervical stop scale 94 is etched into the outside of the catheter guide arm 52 and marks the position of the cervical stop when it is custom-adjusted to each patient prior to insertion.

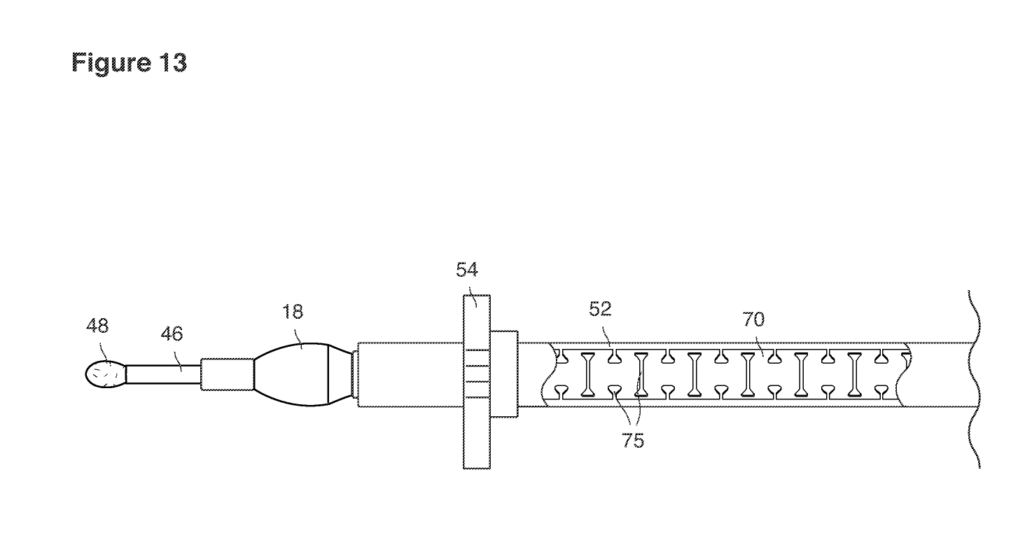

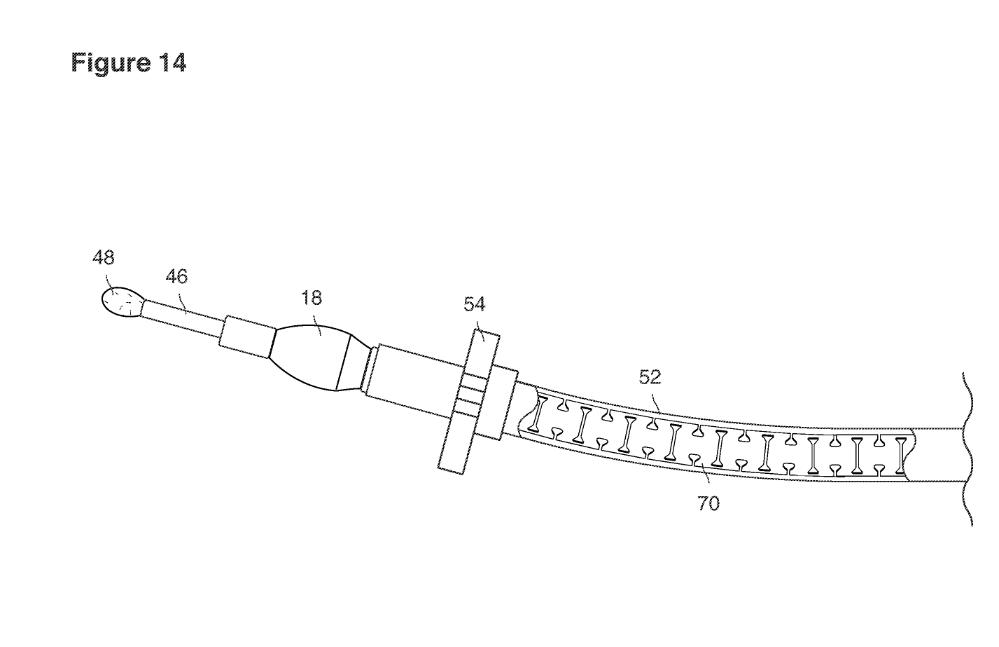

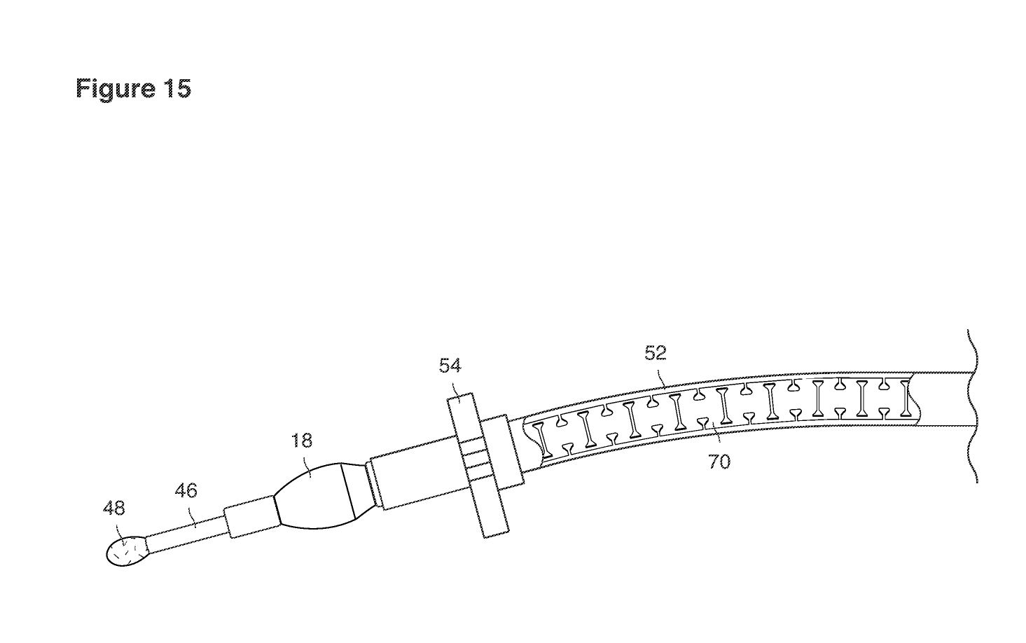

Referring to FIGS. 13-15, the formable tube 70 can be bent into a desired position by the operator to allow the absorbent tip 48 and the suction line 46 of the uterine retrieval device 10 to travel through the cervical canal and into the cervix with minimal discomfort to the patient. The angle can be preset from about 0 to 60 degrees and is customized to individual women in order to accommodate the different anatomical variations of the uterine flexion. FIG. 13 shows the formable tube 70 modified to 30 degrees up, and FIG. 14 shows the formable tube 70 modified to 30 degrees down. The formable tube 70 is made, for example, from Stainless Steel, is coated with polyamide, and includes cut-outs 75.

The outer guide member 38 has an outer diameter in the range of, for example, 6-7 mm, and is made from, for example heat shrink polyolefin or p-bax elastomeric over layer. Inner catheter 40 has an outer diameter in the range of, for example, 3-6 mm, and for example, 3.05 mm, and is made, for example, from stainless steel. Cervical stop 54 has a diameter of, for example, 19.05 mm and is made, for example, from polyamide. The uterine retrieval device 10 is sized for use without anesthesia.

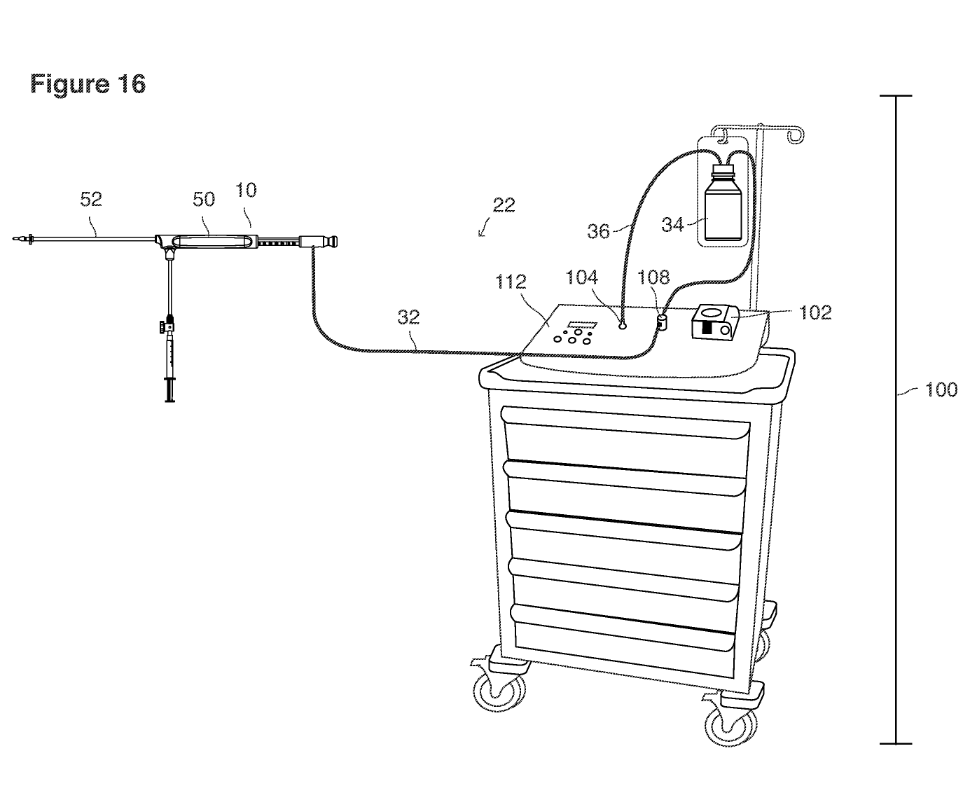

Referring to FIG. 16, the uterine retrieval system 22 can include a control cart 100 that may be used to connect the uterine retrieval device 10 to embryo recovery trap or collection bottle 34, and to control the removal of blastocysts from the uterine cavity. Blastocysts 20 are recovered through the uterine retrieval device 10 and travel to the collection bottle 34 via the suction recovery channel 32. The collection bottle 34 is connected to a vacuum supply connector 104 via the suction line 36 through which suction is applied to suction recovery channel 32. The application and level of suction is controlled by a pinch valve 108. In other implementations, the uterine retrieval device 10 may be used by simply attaching the device 10 to a manually-controlled vacuum system to apply suction. In yet another implementation, the uterine retrieval device 10 may be used without being connected to any vacuum source, instead collecting the blastocysts 20 by recovering them directly from the absorbent tip 48 after removing the device 10 from the uterus.

The control system implemented by the control cart 100 is reprogrammable such that software can be loaded that alters, for example, the frequency of vacuum pressure and the amount of vacuum supplied.

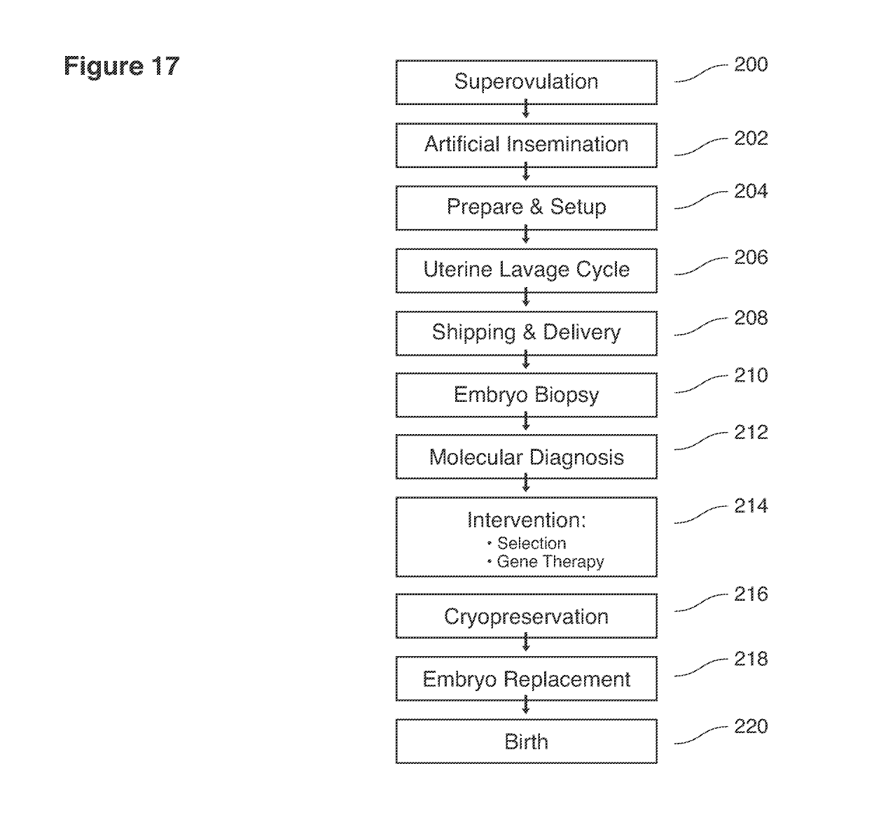

Referring to FIG. 17, the system 22 is used in one or more steps of a procedure that includes superovulation 200, artificial insemination 202, preparation and set up 204, uterine lavage, or retrieval, cycle 206, shipment and delivery of blastocysts recovered during the retrieval process 208, shutdown cycle 222, embryo biopsy 210, molecular diagnosis 212, intervention 214, cryopreservation 216, embryo replacement 218, and ending in the birth 220 of a healthy baby.

Preparatory uterine embryo retrieval, prior to superovulation and insemination, a practice retrieval can be performed (approximately one or two months) before the live procedure is scheduled. In the practice retrieval, measurements are taken (with the assistance of imaging technologies) and the uterine retrieval device 10 is custom fit to enable the anatomy of each patient to be accommodated. Precise imaging of each woman's anatomy utilizes imaging devices, for example, two-dimensional or three-dimensional ultrasound, magnetic resonance imaging, or other imaging technology. The operator determines the optimal position for cervical stop 54 and records the reading on the scale 94, the optimal insertion of stabilizing bar 44 and records the reading on the indicia 82, the angle the uterine retrieval device is to be set at by modification of the formable tube 70, and the amount of inflation of the balloon collar 18 to accommodate the degree of cervical dilation of the patient.

Superovulation is caused in a woman to form multiple corpora lutea that undergo apoptosis and cannot support development of a viable implanted pregnancy following shutdown 222. In-vivo fertilization of multiple oocytes by artificial insemination and/or natural insemination is followed by maturation of the fertilized oocytes to form multiple mature preimplantation embryos that present to the uterine cavity as blastocysts.

To cause superovulation, FSH is delivered to the woman's body. The FSH can be delivered by self-injection. The dosage of FSH is appropriate for induction of superovulation, in vivo fertilization, and embryonic maturation. The FSH is, for example, self-injected daily for 5 to 15 days in the range of 5 to 600 mIU per day. The FSH includes at least one of injectable menotropins containing both FSH and LH; purified FSH given as urofollitropins; recombinant pure FSH; or single doses of long acting pure FSH (recombinant depot FSH), including administering GnRH antagonists to quiet the ovaries while causing superovulation. The GnRH antagonists include receptor blocker peptides. The GnRH antagonists include at least one of Cetrotide 0.25 to 3.0 mg, Ganirelix, Abarelix, Cetrorelix, or Degarelix in which causing superovulation includes administering GnRH including administering a single dose of hCG agonist subcutaneously or snuffed to trigger the superovulation. The GnRH includes at least one of Leuprorelin, Leuprolide acetate, Nafarelin, or Naferelin acetate snuff 117 including administering LH or hCG without GnRH agonist including administering LH or hCG or in combination with GnRH agonist in which impaired (apoptosis) corpus luteum estradiol and progesterone production is supplemented to maintain embryonic viability and maturation by including administrating progesterone and estradiol until recovery of the blastocysts. The progesterone includes at least one of vaginal progesterone, or oral progesterone and the estradiol includes at least one of oral or transdermal estradiol. The progesterone includes Crinone® 1 application per day or Prometrium 200 mg® 3 applications per day or Prometrium 200 mg® 3 oral capsules per day, and the estradiol includes transdermal estradiol patches 400 ug per day or oral estradiol 0.5 to 5.0 mg per day in which blastocyst implantation is prevented by discontinuing administration of estradiol and progesterone starting on the day of blastocysts recovery on the day of retrieval. Desynchronization includes administering progesterone receptor antagonist. The administering includes a single dose of progesterone receptor antagonist (Mifepristone 600 mg) injected into the uterine cavity with a second dose (Mifepristone 600 mg) mg given by mouth one day prior to expected menses. Desynchronization includes administering GnRH antagonist on the day on which the blastocysts are recovered to induce further corpus luteum apoptosis, suppress luteal phase progesterone, and further decrease risk of a retained (on account of blastocysts missed by the intrauterine retrieval) pregnancy. The GnRh antagonist includes Cetrotide 0.25 to 3.0 mg.

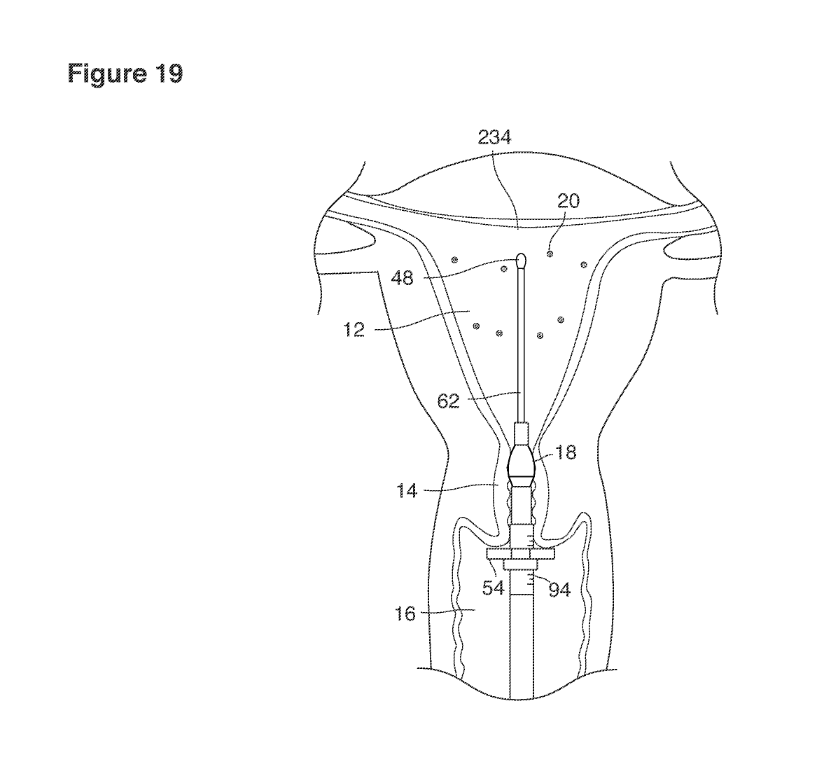

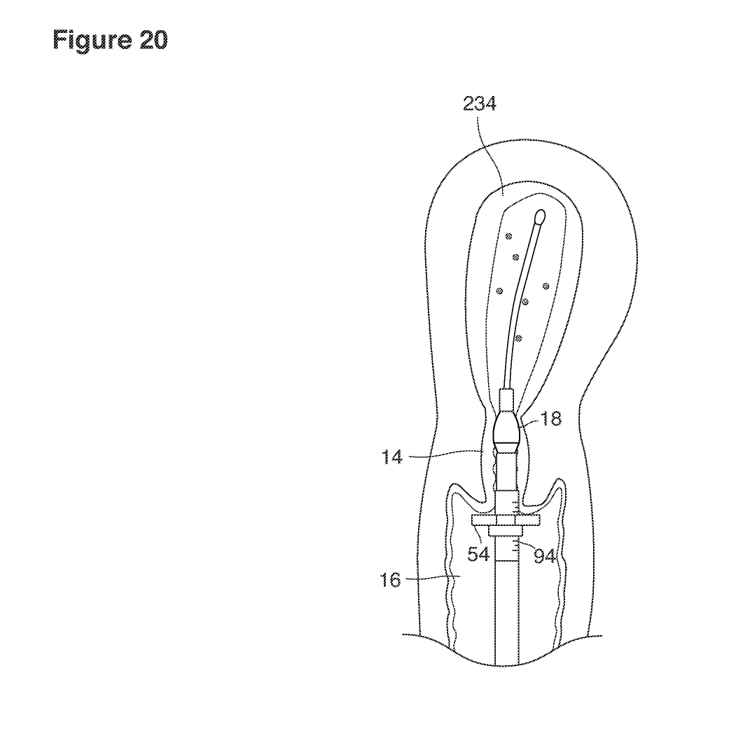

Uterine retrieval is typically performed between 4 and 8 days after the LH dose or LH surrogate trigger that released in vivo the multiple oocytes resulting from the superovulation. Referring to FIG. 19, at the optimal time (most likely day 6), the blastocysts 20 are located between the anterior and posterior uterine walls at approximately the geometric center of the uterine cavity 12. This location is in close proximity to the ultimate site of implantation, which is believed would take place within one day or less after the procedure if the blastocysts 20 were not recovered.

In preparation for the live retrieval, the disposable and reusable elements of the instrument are selected based on the prior measurements and study of the woman's anatomy, and assembled and attached to the pulsing and suction elements, ready for the procedure. The operator sets the cervical stop 54 at the position determined on the cannula that ensures the balloon collar 18 is positioned along the internal cervical os 230. The cervical stop 54 is set relative to the measurement markings on the cervical stop scale 94 that defines the distance from the balloon collar 18, which has been premeasured by the device operator, and is clamped to the catheter guide arm 52.

The operator then shapes the catheter guide arm 52 as predetermined by the operator such that when the uterine retrieval device 10 is placed into the uterus the absorbent tip 48 is positioned for extension along the midline of the uterus. The catheter guide arm 52 is flexible and will hold its shape via internal formable tube 70, and is bent into position to accommodate the position of the uterus relative to the particular woman's body (anteverted, retroverted, cast medially or laterally or any combination therein). The anatomy of the patient in question has been documented in prior exams such that the uterus position information can be used to prepare the uterine retrieval device for embryo recovery.

The uterine embryo retrieval procedure is conducted as follows:

i) Intracervical Insertion: The procedure begins with insertion of the uterine retrieval device 10 into the uterine cavity 12 via the cervical canal 14 through the vagina 16. The uterine retrieval device 10 is inserted until the cervical stop 54 rests against the external surface of the cervix 14 (external cervical os 232) creating a fluid-tight seal, protecting the vagina 16 (FIG. 19). The deflated balloon collar 18 lies at the end of the cervical canal 14 at the entrance to the uterus (internal cervical os 230).

ii) Insufflation: Creation of Cervical Seal: The cervical seal balloon collar 18 may then be inflated (FIG. 1) to provide a watertight seal at the internal cervical os 230 to prevent any leakage around the uterine retrieval device 10. This is done by depressing the syringe 56 until 1.5 cc to 3 cc of fluid, air or liquid, is injected into the balloon collar 18, or until sufficient resistance to balloon inflation is felt by the operator. The stopcock 60 is then closed to ensure the balloon collar 18 remains inflated throughout the duration of the procedure. In some cases, especially for nulliparous women, balloon inflation may not be required to gain a seal at the internal cervical os 230.

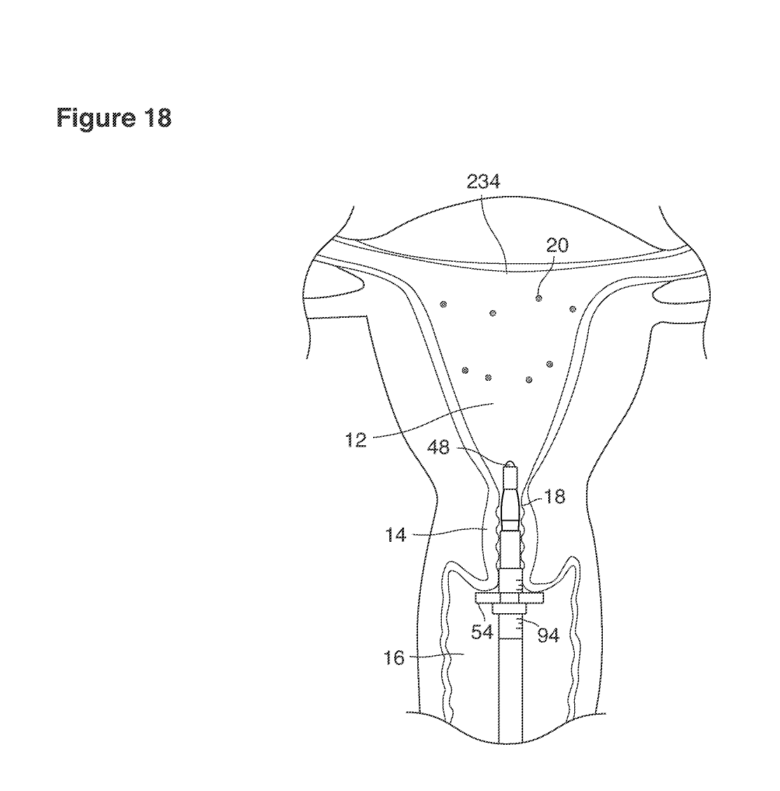

iii) Positioning of Catheter Tip on Inner Surface of Uterus: The final step prior to performing the retrieval is positioning of the absorbent tip 48 on or close to the inner surface of the uterine cavity 12. The operator utilizes predetermined dimension information that specifies the length of the uterus from the external cervical os 232 to the fundus 234 to set the position of the catheter tip 48 as follows: hold the uterine retrieval device using the handle 50; extend the atraumatic tip 48 into the uterine cavity 12 (FIGS. 18 and 19) by pushing the manifold 42 slowly forward until the tip 48 touches the inner wall of the uterine cavity 12. The operator knows when contact with the inner wall has been made when resistance is felt as the outer tubular member 62 is being extended into the uterus while depressing the manifold 42. The uterine retrieval device 10 including its fluid supply and vacuum lines is now in its extended position. By positioning the absorbent tip 48 at the surface of the wall of the uterus, the uterine retrieval device 10 can utilize a swabbing action to help loosen mucus and recover blastocysts in the mucus through sponge-like absorption.

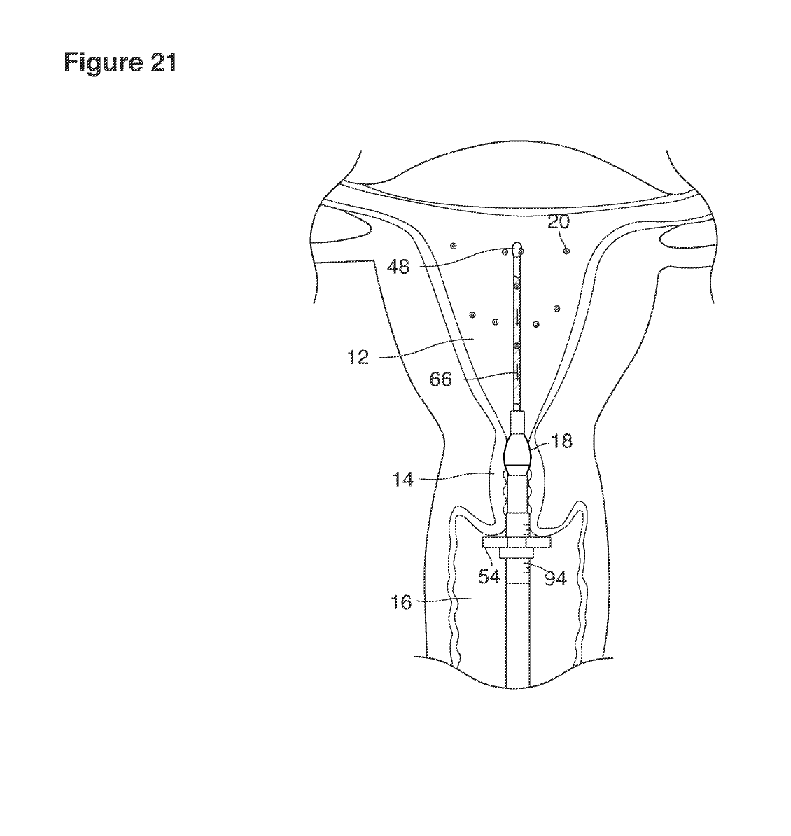

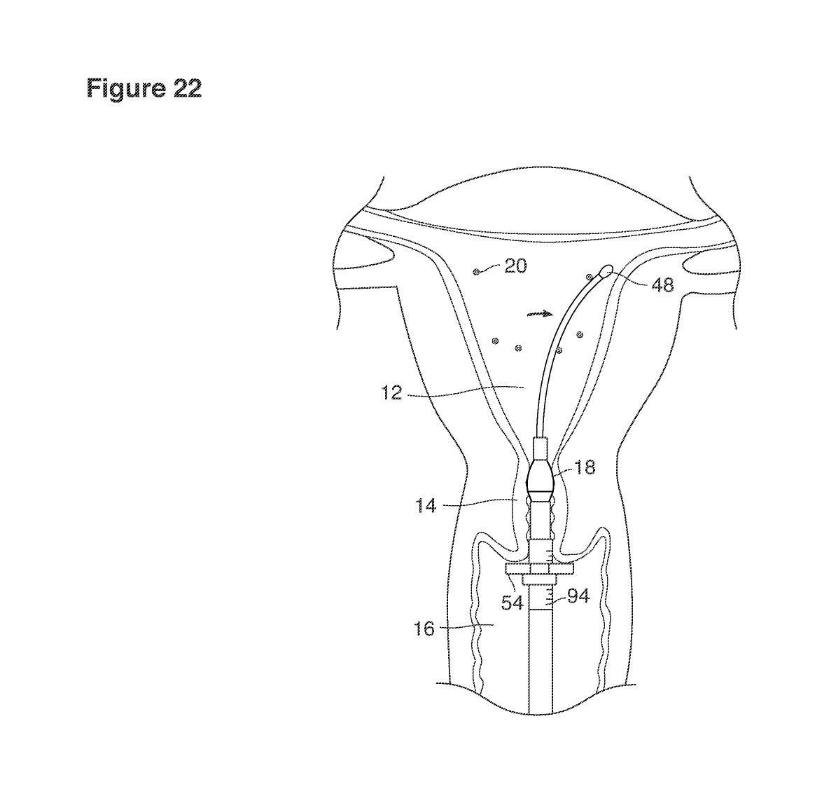

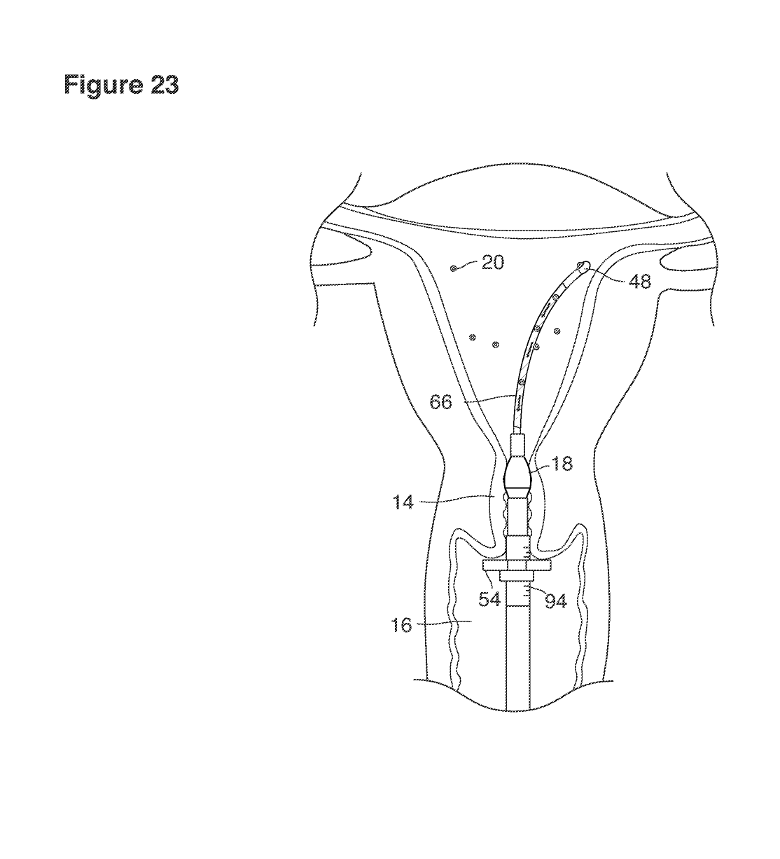

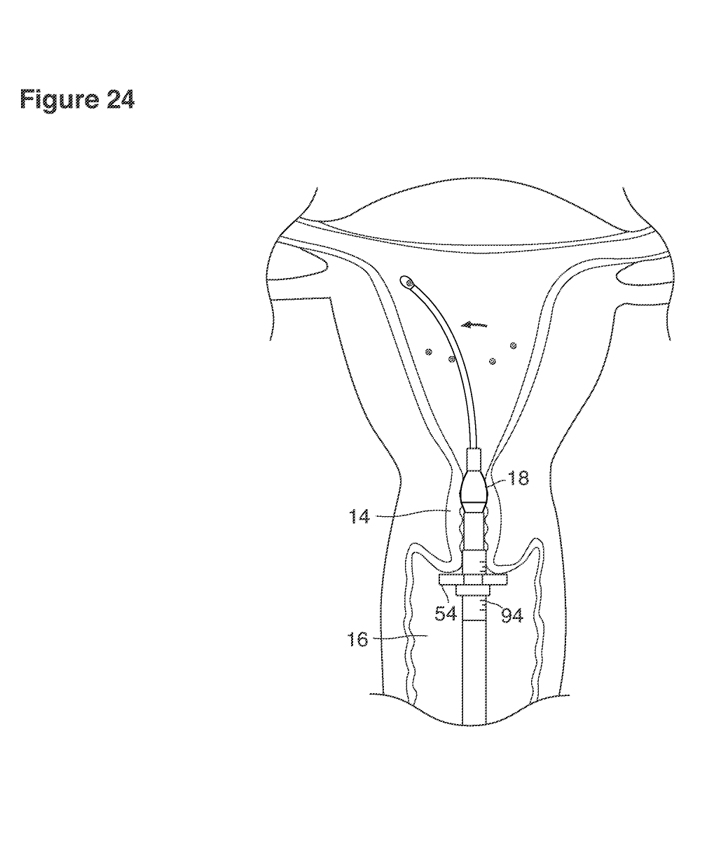

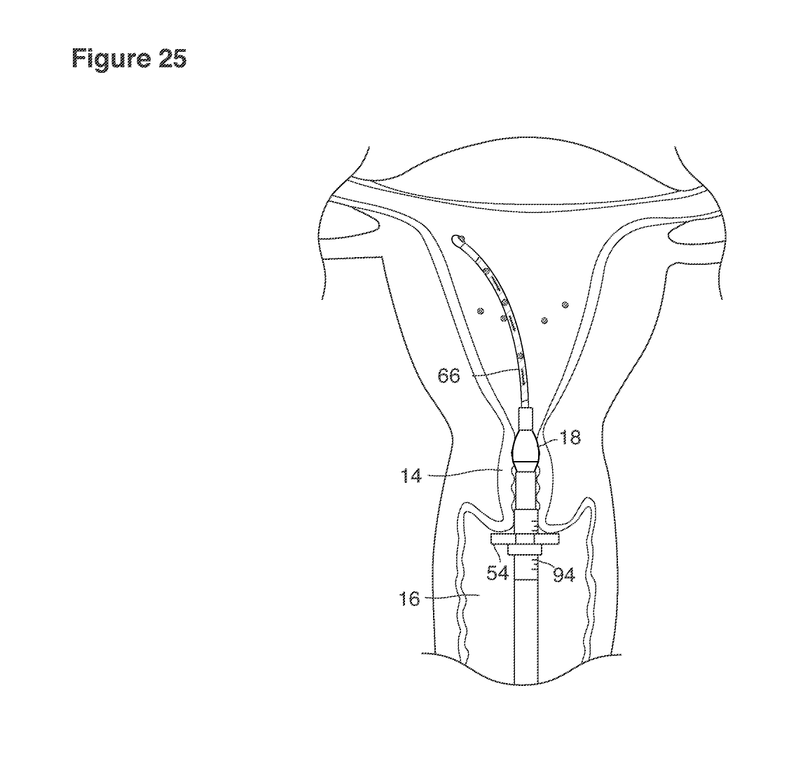

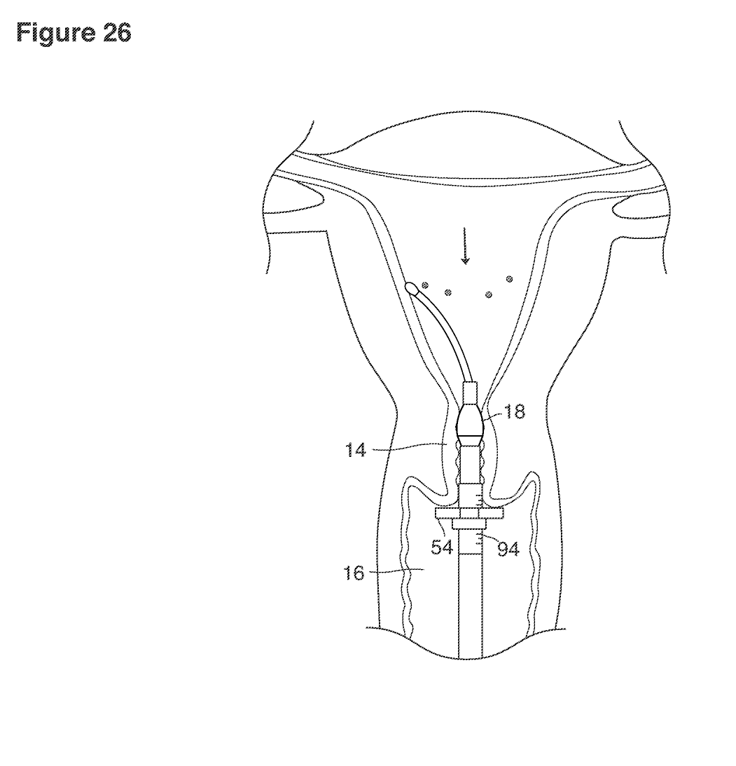

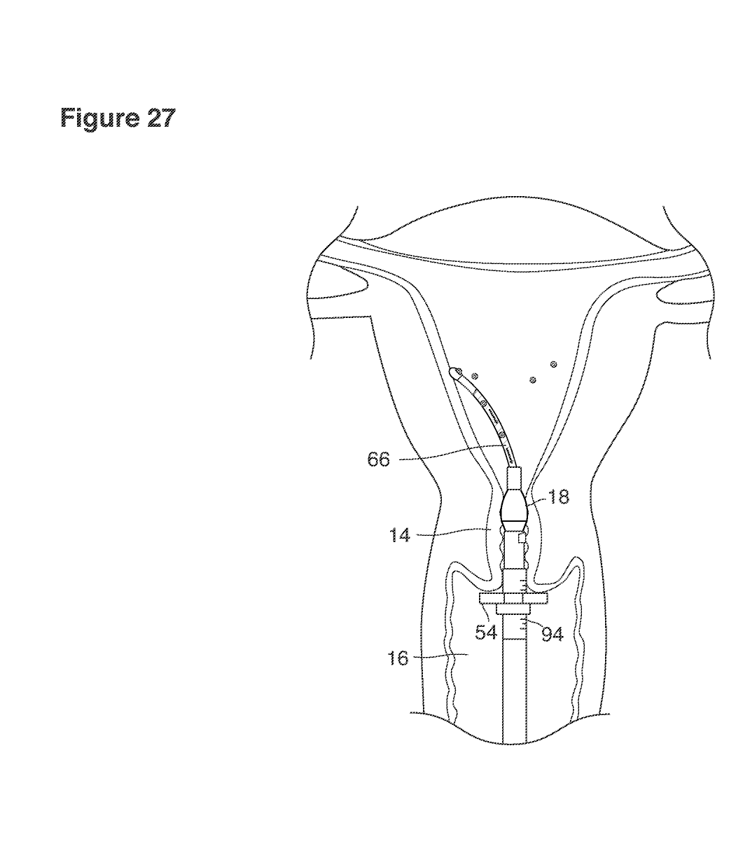

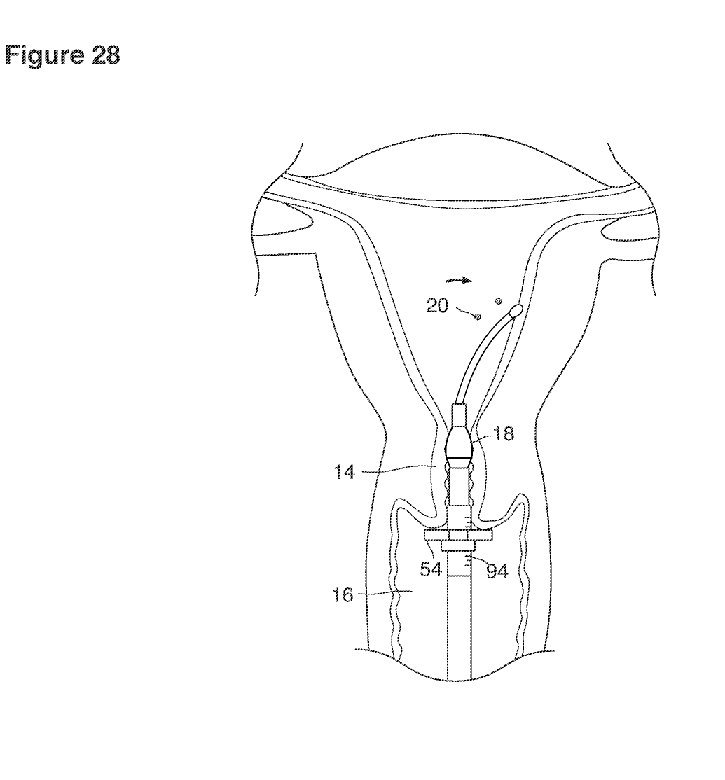

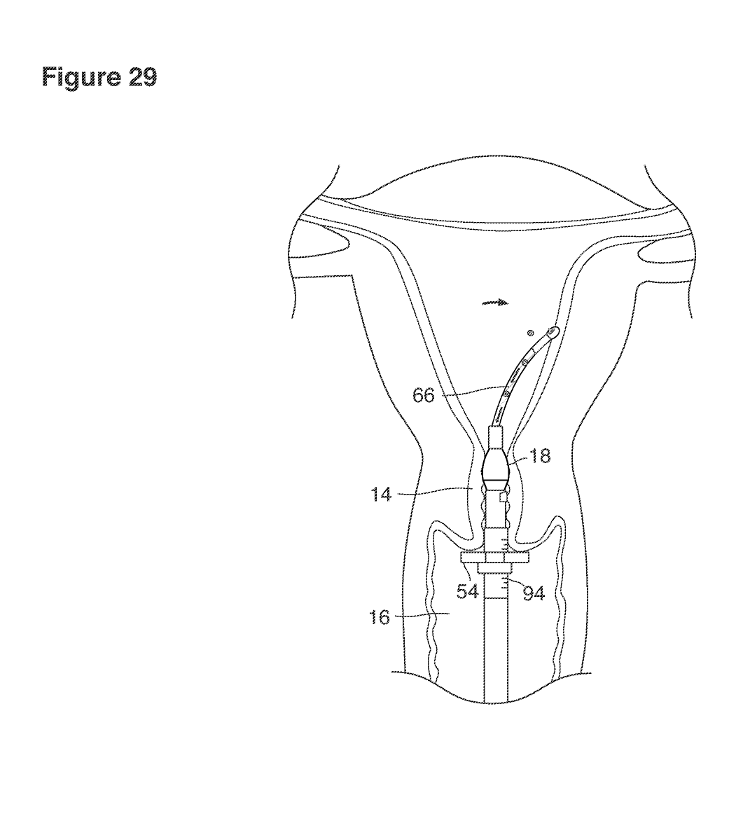

Alternatively, the position of the tip 48 is determined by monitoring the indicia 82 on the stabilizing bar 44. By further pre-bending the outer tube member 62 to a desired shape, the path of the absorbent tip 48 within the uterine cavity 12 can be estimated. iv) Swabbing & Embryo Recovery: After the absorbent tip 48 has been positioned as desired within the uterine cavity 12, vacuum can be applied to the device 10 to suction the absorbed blastocysts 20 away from the tip 48 through the outflow lumen 66 (FIG. 21). In some cases, the vacuum applied can help enhance the absorbing effects of the tip 48. Subsequent stages of embryo recovery (FIGS. 22-29) can be performed by manipulating the absorbent tip 48 along the wall of the uterus by, for example, rotating, extending, and/or retracting the outer tubular member 62 (FIGS. 22, 24, 26, and 28). After repositioning the absorbent tip 48, the blastocysts 20 can be recovered as described above.

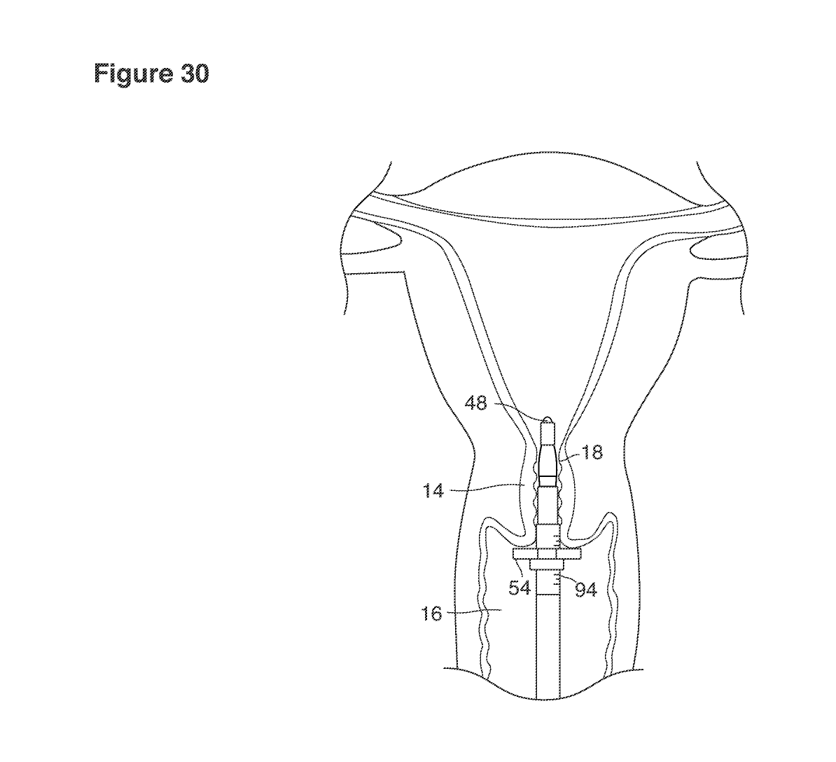

v) Removal of Uterine Retrieval Device: The operator removes the uterine retrieval device as follows (FIG. 30): pull the manifold 42 away from the handle 50 to retract the inner catheter 40 into the outer guide member 38; deflate the balloon collar 18 by opening the stopcock 60 and retracting the syringe 56 to 0 cc; the uterine retrieval device 10 is then slowly removed from the cervix 14.

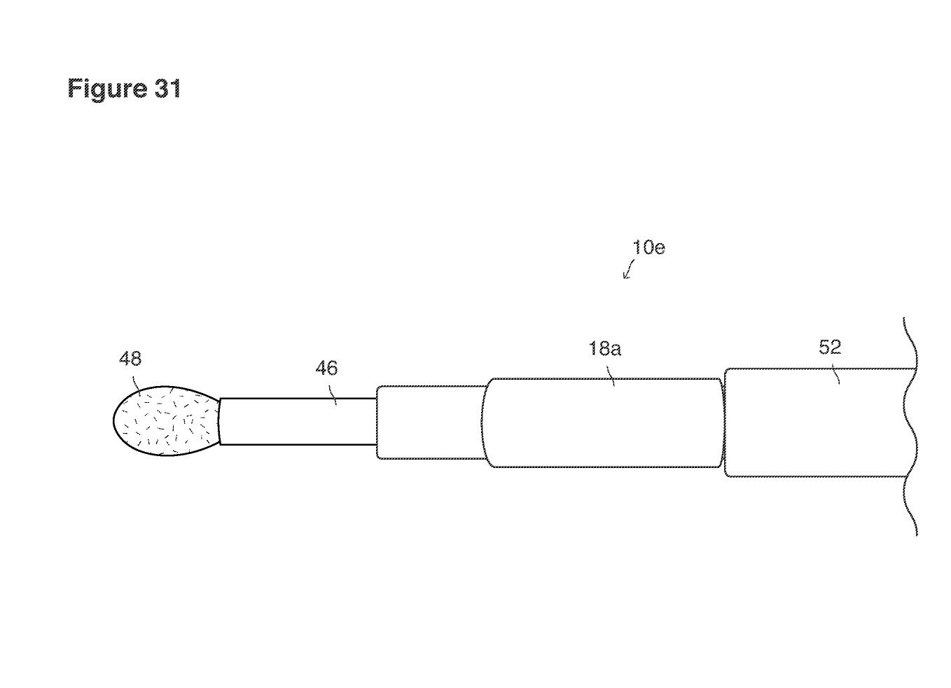

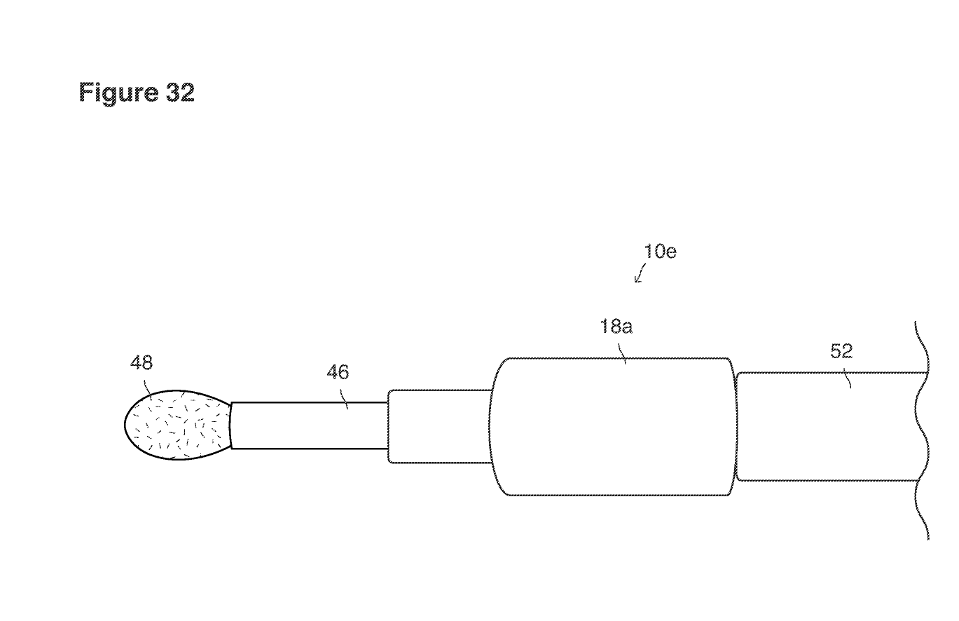

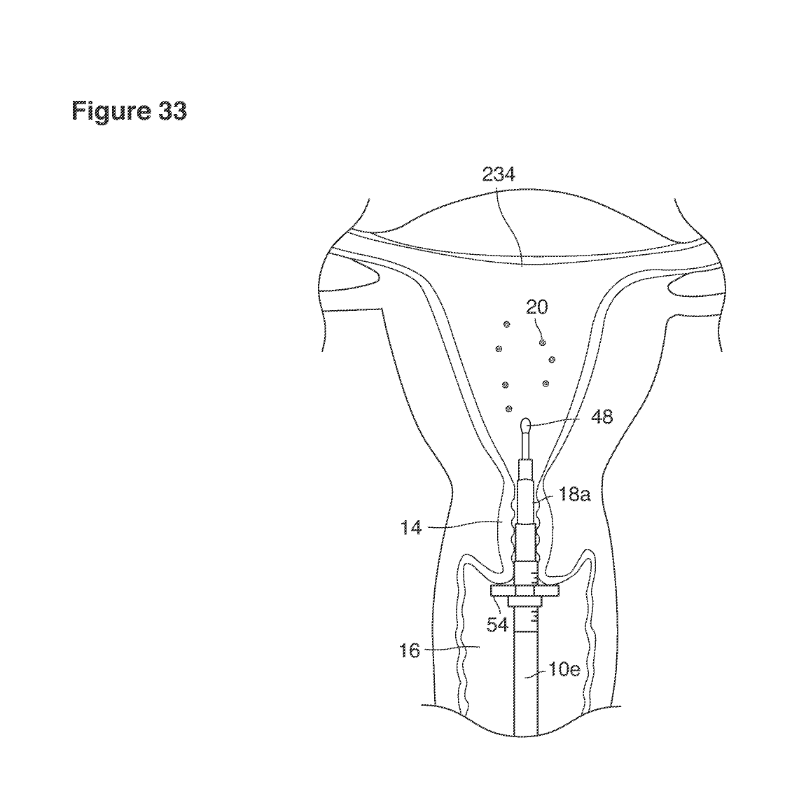

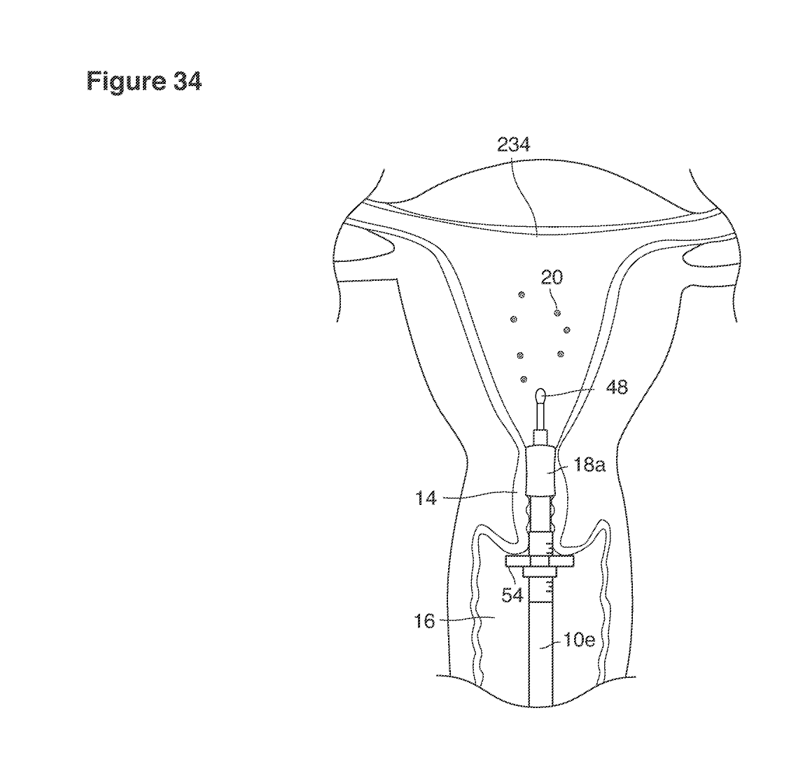

Referring to FIGS. 31-34, a uterine retrieval device 10e includes an activatable seal in the form of expandable foam 18a. The foam 18a is compressed prior to insertion and expands within the cervix to seal the uterine cavity from the external environment, as illustrated in FIG. 34.

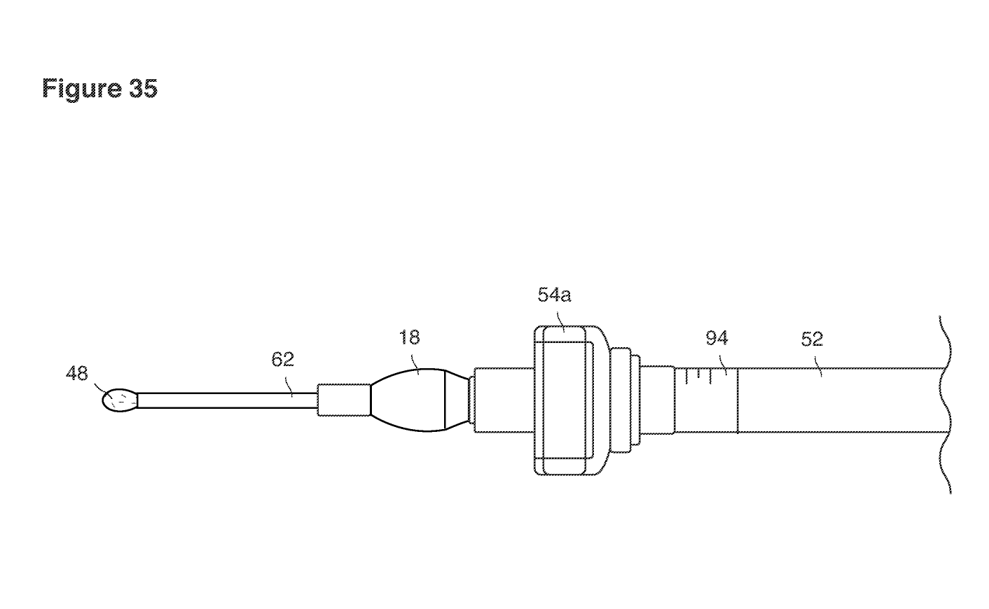

In some implementations, the cervical stop 54 can be replaced with a cervical cup 54a (FIG. 35). The position of the cervical cup 54a can be adjustable relative to the balloon collar 18 along the cervical stop scale 94 on the guide arm 52. The position of the cervical cup 54a defines a dimension corresponding to a distance from an opening of the cervix at the vagina (the external cervical os) and an opening of the cervix at the uterus (the internal cervical os). The position of the cervical cup 54a may be fixed in position relative to the guide arm 52 prior to insertion of the device 10. The cervical cup 54a can be made from a flexible material, such as polyamide, and can have inner and outer diameters in the ranges of, for example, 3-9 mm and 6-12 mm, respectively. In some cases, the cervical cup 54a may be fixedly attached to a distal end of the guide arm 52. In this case, the relative position of the cervical cup 54a to the balloon collar 18 may be adjusted by extending and retracting the support member 74 relative to the guide arm 52.

In some implementations, vacuum may be applied to the cervical cup 54a to attach and seal the cup 54 to the external cervical os. The operator can then pull on the uterine retrieval device 10a to straighten the woman's uterus.

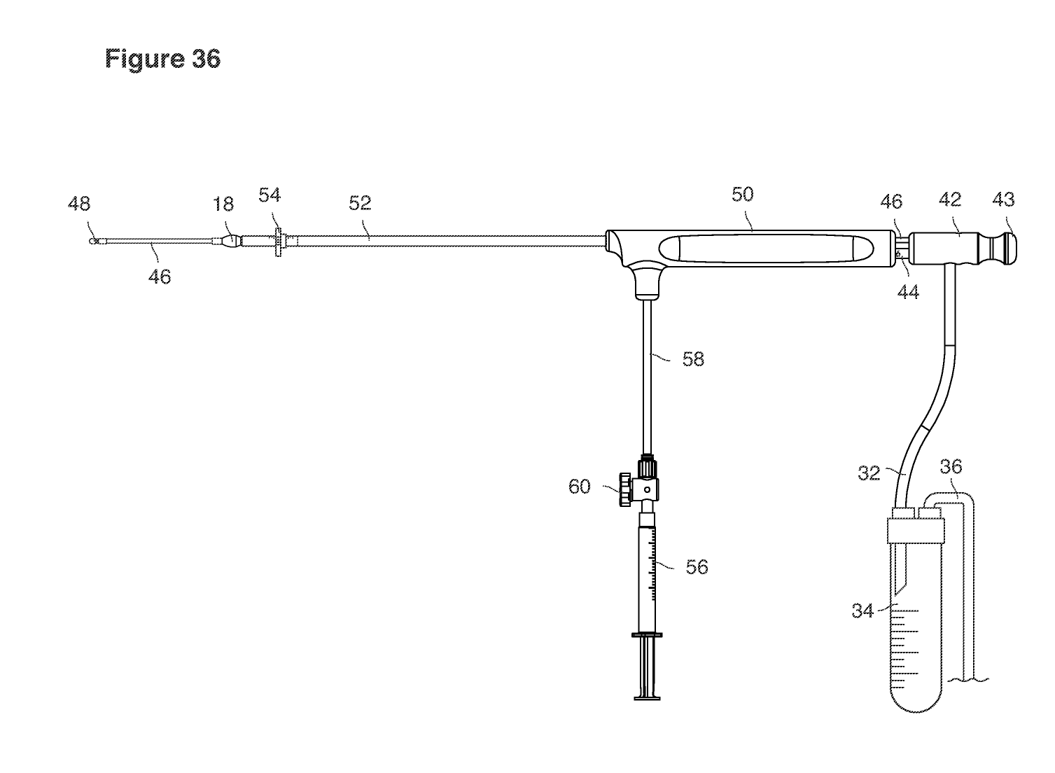

Referring to FIG. 36, rather than having the collection bottle 34 mounted to the cart 100, as shown in FIG. 16, the collection bottle 34 can hang off the device 10 with the suction line 36 running to the cart 100.

A number of embodiments of the invention have been described. Nevertheless, it will be understood that various modifications may be made without departing from the spirit and scope of the invention. Accordingly, other embodiments are within the scope of the following claims.

What is claimed is:

1. A device for recovering one or more blastocysts from a uterus of a human, comprising:
an outer guide member for insertion into a cervical canal of the human, the outer guide member including a distal portion with an activatable seal for isolating the uterus from the external environment, the outer guide member defining a lumen having a longitudinal axis; and
an inner catheter located within the lumen and slidable along the longitudinal axis of the lumen relative to the outer guide member, the inner catheter having a distal tip positionable distally of the seal to extend into the uterus, the distal tip including a semi-permeable absorbent head,
wherein the device defines a distal suction port for aspirating blastocysts from the uterus through the absorbent head.

2. The device of claim 1, wherein the inner catheter defines the distal suction port.

3. The device of claim 1, wherein the absorbent head has an atraumatic shape.

4. The device of claim 1, wherein the inner catheter is steerable.

5. The device of claim 4, wherein the inner catheter includes a memory-retaining material, and wherein, upon extension into the uterus, the inner catheter is configured to be steered by bending according to a pre-bent shape of the memory-retaining material.

6. The device of claim 1, wherein the activatable seal is a balloon collar.

7. The device of claim 1, wherein the activatable seal is an expandable foam.

8. A system for recovering one or more blastocysts from a uterus of a human, comprising:
a device, comprising:
an outer guide member for insertion into a cervical canal of the human, the outer guide member including a distal portion with an activatable seal for isolating the uterus from the external environment, the outer guide member defining a lumen having a longitudinal axis; and
an inner catheter located within the lumen and slidable along the longitudinal axis of the lumen relative to the outer guide member, the inner catheter having a distal tip positionable distally of the seal to extend into the uterus, the distal tip including a semi-permeable absorbent head,
wherein the device defines a distal suction port for aspirating blastocysts from the uterus through the absorbent head; and
a controller programmed to apply vacuum to the device from a vacuum source remote from the device.

9. The system of claim 8, wherein the controller includes a pump for applying the vacuum.

10. The system of claim 8, wherein the controller includes electro-mechanical means for controlling the vacuum.

11. The system of claim 8, further including an embryo recovery trap for receiving the blastocysts.

12. A process for recovering one or more blastocysts from a uterus of a human, comprising:
placing a device trans-vaginally into the cervical canal, the device including an outer guide member and an inner catheter located within the outer guide member, the outer guide member including a seal for isolating the uterus from the external environment;
advancing the inner catheter relative to the outer guide member positioning a distal region of the inner catheter within the uterus; and
applying a vacuum to the uterus to aspirate blastocysts from the uterus through an absorbent head of the inner catheter.

13. The process of claim 12, wherein placing the device includes locating the seal in the cervical canal.

14. The process of claim 13, wherein locating the seal includes locating the seal between the internal cervical os and the external cervical os such that the seal does not extend into the vagina or the uterus.

15. The process of claim 12, wherein advancing the inner catheter includes swabbing an inner surface of the uterus with the absorbent head.

16. The process of claim 12, wherein advancing the inner catheter includes positioning the absorbent head proximate an inner wall of the uterus.
